(19) United States
(12) Patent Application Publication
(10) Pub. No.: US 2026/0157644 A1
(43) Pub. Date: Jun. 11, 2026

(54) Method and means to make use of multiple simultaneous blood pressure measurements for detection of arterial obstruction with electronic and traditional type measurements

(71) Applicants: Sergio Lara Pereira Monteiro, Los Angeles, CA (US); Sabaka Bumerangue, Los Angeles, CA (US)

(72) Inventors: Sergio Lara Pereira Monteiro, Los Angeles, CA (US); Sabaka Bumerangue, Los Angeles, CA (US)

(73) Assignee: Sergio Lara Pereira Monteiro, Los Angeles, CA (US)

(21) Appl. No.: 19/418,848

(22) Filed: Dec. 12, 2025

Related U.S. Application Data
(60) Provisional application No. 63/729,893, filed on Dec. 9, 2024.
(60) Provisional application No. 63/733,976, filed on Dec. 13, 2024.
(60) Provisional application No. 63/781,978, filed on Apr. 1, 2025.
(60) Provisional application No. 63/800,406, filed on May 5, 2025.

(51) Int. Cl. A61B 5/022; A61B 5/021; A61B 5/0215; A61B 7/04

(52) U.S. Cl. CPC A61B 5/02208; A61B 5/02141; A61B 5/0215; A61B 7/04

(57) ABSTRACT

We disclose a device to detect obstructions located in the inner part of conduits for fluids. One application is for the detection of atherosclerosis, or internal obstructions in the inner walls of arteries, using sphygmimanometers, photoplethysmographaphs, positioned at symmetrical positions on the body of the animal, which usually is a human. A modification uses the propagation time of the pressure wave of the blood pumping events, from the heart to some extremity point, as an arm or a leg. Finally we also disclose a similar device, using the time differences between the arrival of the heart pulsations on the right and on the left sides of the body of a patient. The device to measure the blood pressure may be a sphygmomanometer, or may be a FitBit, or may be a oximeter, or may be an invasive pressure detector inserted in an artery, or any other device.

### BACKGROUND OF THE INVENTION

### Field of Invention

This invention is related to the art of detecting obstructions to the flow of fluids through conduits, as tubes and pipes and the like, with application to arterial obstructions in the arteries in general, including the larger arteries exiting the heart, and their subdivisions into smaller arteries towards the body periphery.

### BACKGROUND

### Discussion of Prior Art

The accumulation of debris inside pipes, tubes, and other means to convey fluids from one location to another is detrimental to the flow, because said debris obstruct the fluid flow, or, in technical parlance, it causes decreases in pressure and speed of flow. The former, pressure drops, requires extra energy to maintain the fluid flow, the latter, speed drops, decreases the efficiency of the fluid transportation. It is therefore necessary to discover the existence of debris inside the fluid conduits, which perhaps allows the removal, or cleaning of the conduits, when desired, if physically possible. One of the methods used for the discovery of the existence of debris inside the conduit of fluids is the measure of pressure at several position along the fluid motion, because the existence of debris causes a pressure drop.

We are going to use as an example the flow of blood (the fluid) inside arteries (the pipe), which is one medical application that has much importance, because the accumulation of debris inside the arteries (or veins) causes medical problems, including the death of the debris accumulator, an event which he usually tries to avoid. The accumulation of fat and other cells inside the arteries, mostly often referred as atherosclerosis, is considered to be a medical problem which may lead to death of the animal affected by it. It is so because it decreases the required blood flow, which is necessary to bring nutrients to the cells, and also because such fat deposits may get lose from their location, moving through the arterial blood system until reaching a smaller artery, beyond which it can not progress, and likely causing a sudden and catastrophic blood supply decrease, perhaps in the brain or in the miocardium.

This problem lead to the search of methods to discover the existence of debris accumulation inside the arteries, or, using medically terminology, the existence of atherosclerosis. One such method is the full body MRI scanning. The condition can be visualized with MRI scanning, but MRI being such a complex and expensive procedure, it is difficult to imagine MRI being used as a population scanning practice, similar to the females searching for lumps on their breasts, or dentists cleaning of the teeth while visually looking for cavities. Consequently, MRI full body scanning will continue to be used, because it is effective, but other, alternative methods are necessary, due to the cost and complexity of MRI scanning. Then, to make the situation worse, the sector of the population that are more at risk of atherosclerosis is the older part of the population, which is more likely to use either a heart pacemaker or other type of electrical stimulator, as brain, spine, etc., which are generally incompatible with MRI.

Another possibility for scanning for atherosclerosis is to look for the time delay of the heart beat measured on one side of the body of an animal, compared to the equivalent position on the other side of the body of the same animal. For example, a medical person may use fingers of two hands, perhaps the thumb finger of each hand, to detect the wrist pulsation of the heart at the radial arteries (at the wrist) on the left and the right side of an animal, usually a human, looking for time delays. On a healthy person, with his arteries clean from any internal deposits, the heart beating at the right and left sides occurs concomitantly when both sides are measured at the same location, say, the wrist. We are here also assuming that his arteries are, as normally they should, of the same inner diameter on each side of his body, which is the case for the vast majority of the animals, including humans. Minimal variations of the inside diameter of the arteries going to the right arm and the left arm are expected, many within the normal expectancy of measurement uncertainty, and would be disregarded, even if just above the measurement uncertainty. If, on the other hand, there is an accumulation of fat tissues, or any other debris in the inner side of the artery on the right arm, say, the brachial artery, at the upper part of the arm, this causes a pressure drop at the position of this partial obstruction, which, in turn, decreases the speed of the blood flow from the obstruction point on (lower blood pressure causing a lower propagation speed of the blood), which, in turn, causes that the heart beating detected at the radial artery on the right side (with the partial block a little above) occurs later in time than the equivalent heart beating detected on the left arm also at the radial artery but on the symmetric position, the left side. In other words, the heart beating detected at the radial artery on the left side (the “good” side) occurs prior to the beating detected at the radial artery on the right side (the “bad” side). This is often used by physicians and nurses at many medical facilities. Recapitulating, an internal obstruction of an artery that serves the right arm, normally causes both a pressure drop of the blood flow through the right arm and also a delay of the arrival of the beating, which is an indication of an internal obstruction to the blood flow.

All along, while it is easier to detect time delays on the wrist heart beat arrival, the arteries that are involved on the blood supply to the wrist are short. The main health problem is on the arteries that supply blood below the arms, blood for the lower part of the body, which are not measured by time delays at the wrist. For blood supply below the arms, which is the most common location of blood supply problems, the medical person needs to search for time delays at the legs, ideally at the lower part of the legs, which is more difficult to detect than the equivalent search for delays at the wrist. All these are problems for the medical person, which are asking for a solution. Our patent application is a method and a means to solve this problem - among other problems.

Another method, a different method, is the measurement of the blood pressure on both arms (or legs, etc., any two symmetric locations on the body), because the obstruction to the blood flow causes a pressure drop, which then causes that the blood pressure measured at any location downstream is lower on the side of the obstruction than on the non-obstructed side. The measurements are taken at symmetrical locations, say, just above the elbow being the most common location, on each side. In this case, of an atherosclerosis on the right side, perhaps at the subclavian artery, the blood pressure measured on the right arm (the “bad” one) is lower than the blood pressure measured on the left arm (the “good” one). This is the application of the invention described here, which will be described for blood flow, but is valid for any other fluid flow progressing along any pipe or tube.

Another point that needs to be considered here is that each heart beat is characterized by a different strength, or squeezing, or applied pressure - heart beatings are not the same! Or, saying it from a larger perspective, each heart beat is characterized by a different power, or strength, or vitality. Some heart beatings are stronger, some heart beatings are weaker, and there is no pattern on their occurrences. It follows that each heart beating is characterized by its own systolic (high) and its own diastolic (low) blood pressure values. In other words, the change, or variation of contraction strength of the heart, is a phenomenon occurring from beat to beat, each beat being characterized by a different strength. This, in turn, introduces a difficulty of using the difference of blood pressures (systolic/diastolic) as an indicator of a possible partial blockage on the artery, because the standard way to do it, is to measure the blood pressure on one side, then, LATER, to measure the blood pressure again on the other side. But if a blood pressure is measured to be lower on the right side, when compared with the blood pressure on the left side, it could have been caused by an obstruction on the right side, but it could also be caused by a weaker heart beating at the time the blood pressure is measured on the right side! This problem need to be solved, if one is hoping to use the blood pressure measurements as a possible indicator of arterial obstructions—which is the objective of the invention we describe here: we disclose a method and several means to measure the blood pressure at symmetric locations at the same time, instead of one side than later the other side.

Indeed, the method of measuring the blood pressure on both sides is well known in the medical community, but it is often not used because of its inconsistency—though other reasons are also at play, particularly that there is more money to be made by the big medical companies with the sale of an MRI machine than with the sale of such a simple device as a sphygmomanometer or a photoplethysmographer, which are the hard-do-pronounce names of two of the ordinary and simple devices used all over to measure the blood pressure at the arm of humans (for the sphygmomanometer case), or at several other places (for the photoplethysmographer case), or a few other blood pressure measuring devices that are around. The inventors are aware of this financial obstacle, but the commercialization of the device is not part of our invention, and the inventors are involved with the technical side, exclusively, and not involved with the later commercialization of the invention.

Recapitulating, our invention solves the problem of detecting atherosclerosis from the differences in blood pressure measured at two symmetric points of the body of the animal when the blood pressures are taken in sequence, not at the same time. Our invention discloses a method to take the blood pressure at the two or more different sites at the same time, therefore solving this problem of the difference in blood pressure from beat-to-beat. Our invention also discloses a method and a means to measure that time-of-arrival of the heart contractions at symmetric locations on the body of the animal, which is a separate, different and independent method of detecting blood clots in the arteries. Finally, our method is as easy to apply at the extremity of the legs, as it is to apply at the extremity of the arms, or even to the head, say, at the right and left earlobes. It is a most wonderful invention, really! I like it!

### BACKGROUND

### Discussion of Prior Art

### Definitions

We start this section with the definition of the most important terms we use, in order to comply with the USPTO requirement, as per 35 U.S.C. 112(a) or 35 U.S.C. 112 (pre-AIA), first paragraph, which requires the specification to be written in “full, clear, concise, and exact terms.” We define here the use of “exact terms” as we use, so as not to leave room for misunderstandings of the meaning of our words as used in this document. Firstly here we specify some key terms we use, then some of the abbreviations used in the figures, with their precise definition too.

Throughout this patent specification the use of the word “comprising” includes the case of “consisting only of”.

Antecubital fossa: see cubital fossa.

Atherosclerosis: this is a condition in which fatty deposits, called plaque, build up in the inner lining of the arteries.

Bluetooth: a short-range wireless technology standard that is used for exchanging data between fixed and mobile devices over short distances and building personal area networks (PANs). (https://en.wikipedia.org/wiki/Bluetooth#Etymology, accessed 250429)

Clock signal: in electronics, particularly in digital electronics, it is an alternating signal, usually, but not necessarily, an alternating electric potential (voltage), also usually, but not necessarily a square wave, which is used by other electrical and electronic circuits to determine their working pace, which usually is to give beating orders to do each step as needed.

Conduit: we use this word as a general term to refer to a path through which a fluid flows, as a water pipe, an oil pipeline, an artery of a vein, etc. As used in this document, “conduit” includes both rigid and flexible fluid pathways, as pipes and hoses.

Cubital fossa: It is an area of transition between the anatomical arm and the forearm. It is located in a depression on the anterior surface of the elbow joint. It is also called the antecubital fossa because it lies anteriorly to the elbow (Latin cubitus) when in standard anatomical position.

Cuff: copied from a dictionary: A “cuff” is most commonly a band of fabric at the end of a sleeve or trouser leg, but it can also refer to an inflatable band for measuring blood pressure, a style of bracelet, or a blow with the hand. →We here will use the word with the second dictionary meaning: “. . . an inflatable band for measuring blood pressure, . . . ”

Forearm: is the part of the upper limb that extends from the elbow to the wrist. It is composed of two bones, the radius and the ulna, which are connected by an interosseous membrane. Cf with upper arm, which is the region between the elbow and the shoulder.

Anatomists call the upper arm simply as arm. They use the word arm only for the portion between the shoulder and elbow. The region from elbow to wrist is called forearm.

Photoplethysmography: https://en.wiktionary.org/wiki/photoplethysmography->The recording or study of photoplethysmographs

https://en.wikipedia.org/wiki/Photoplethysmogram->A photoplethysmogram (PPG) is an optically obtained plethysmogram that can be used to detect blood volume changes in the microvascular bed of tissue

Photoplethysmography: Definitions of

https://www.google.com/search?client=ubuntu&hs=BPQ&sca_esv=217f17c28f4e2843&channel=fs&q=Photoplethysmogram+bl ood+pressure&sa=X&ved=2ahUKEwj2po-HuYGNAxXSEUQIHXh8BKwQ1QJ6BAh6EAE&biw=1812&bih=908&dpr=1

### AI Overview

Photoplethysmography (PPG) is a non-invasive optical technique that can be used to indirectly estimate blood pressure. PPG measures changes in blood volume in the microvascular tissue bed, and these changes are related to arterial pressure fluctuations. While PPG cannot directly measure blood pressure like a cuff-based device, it can provide valuable information about blood pressure trends and potentially aid in the early detection of hypertension

How photoplethysmographs work:
PPG Measures Blood Volume Changes;
PPG sensors detect changes in the amount of light absorbed or reflected by blood vessels in the skin. These changes are directly related to the volume of blood in the microvasculature;
Indirect Blood Pressure Estimation;
By analyzing the PPG signal and its features (like the pulse transit time, the shape of the waveform, and other parameters), it's possible to estimate arterial blood pressure.

In summary, PPG is a promising technology for non-invasive blood pressure monitoring and can be used in wearable devices for continuous and potentially accurate BP estimation, although it requires careful validation and calibration

Plaque: In medicine, it is a small, abnormal patch of tissue on a body part or an organ. Plaques may also be a build-up of substances from a fluid, such as cholesterol in the blood vessels.

Pulsating: to expand and contract rhythmically, as the heart; beat; throb. Synonyms: pulse. to vibrate; quiver.

Two possible uses of pulsating:
1) characterized by a strong regular rhythm; throbbing, as in “a pulsating bass line”
2) very exciting, as in “Lazio edged Juventus 2-1 after a dramatic finish to a pulsating semi-final”

For us here, pulsating is used as a variation of 1), an almost regular rhythmic beatings, which occurs at time delays that vary “little” from one event to the next.

Sphygmomanometer: big word for the ordinary device used to measure the blood pressure, usually just above the elbow, in the upper arm, of a person. Virtually everybody has seen these devices.

StupidfyingPhone: or stupid phone: a wireless communication device that works as a traditional telephone but no wires connecting to a hear and talking piece, nor any wire to the outside. It is marketed with a wrong name of Smart Phone—which is a rather undeserved name, given that it is not smart at all, and on top, it makes the human users complete imbeciles.

Upper arm: also known as the brachium, is the area of the arm between the shoulder and elbow joint. Cf. With forearm, which is the region between the elbow and the wrist.

### Text

Blood pressure measurement is likely to be the most important measurement for health screening among any other measurement, if compared with a device of the same simplicity, cost and easiness to use. Granted that it is not able to detect all diseases, but then, no known, existing device is able to detect all diseases, so it is not worse on this account than other devices, and then, for its cost, availability and easiness to apply, it is considered by many public health people to be the most efficient device available for the practice of medicine. It follows that it is worth to improve on this technique—if possible. Our wonderful invention is one such improvement on the blood pressure application for public health and screening for health problems.

Our invention is a method and a means to increase the information obtained by measuring blood pressure and the time of arrival of the heart beatings at different parts of the body, to offer information about the state of health of a person. Our invention is based on blood pressure measurements. In fact, some aspects of our invention are old stuff, while our invention simply adds simultaneity of measurements to the old stuff. Naturally that the simultaneity of measurements is not trivial at all, it fully deserves the patent, even if in the absence of the simultaneity it is all old stuff. Also, with the traditional method of measuring blood pressure, namely, the inflatable cuff applying pressure, usually at the arm, just above the elbow, to stop the blood flow, alone and by itself does not lends itself to a simultaneous pressure measurement, and only after the introduction of a connecting tubing (see figure FIG. 1), does our new, interesting and fascinating method becomes feasible. There are many other methods to measure blood pressure, of which we just mention here the photoplethysmography and the direct, invasive method of inserting a pressure measuring device inside the blood vessel. Our invention works well with any method to measure the blood pressure.

One of the old-time, traditional methods used by physicians to detect obstructions in the blood vessels was to measure the blood pressure on both sides of the body—and at symmetrical positions, left-and-right sides. This equality of the blood pressure measured on the right and left sides, and at the same relative position, is intuitive to expect, because most animals are symmetric on their features, and consequently the blood pressure ought to be symmetric as well. If the patient were symmetric, including the internal diameter of the blood vessels and also including the elasticity and thickness and other properties of the blood vessels, than the blood pressure would decrease by the same amount as the blood advances on either side, and consequently, when the medical practitioner measured the blood pressure at symmetrical positions on the body of the patient, then the blood pressure would be equal; same blood pressure on the right as on the left side of the patient when measured at the same position on either side of the body. If, on the other hand, the patient were under a process of atherosclerosis, that is, accumulation of fatty tissues inside the blood vessels, then, excluding the unlikely case that these atherosclerosis would occur exactly the same way on both sides, the blood pressure would be different, when measured at the same relative position on the right and on the left side of the body of the patient, because as the blood flows through the blood vessels that are obstructed (atherosclerosis) where, at which obstructions, there would occur an extra pressure drop to the blood flow at the constrictions, causing a lower measurement of blood pressure downstream from said obstruction. Blood pressure is usually measured just above the elbow, and consequently this method was applied more at the arms then at the legs or any other location on the body of the patient. This method would have been more informative if the blood pressure at the legs were compared, as opposed to compare the blood pressure differences at the arms, because then the pressure measurement would include abdomen blood vessels obstructions as well, which are not detected by measuring the blood pressure at the arms. For many reasons, the blood pressure at the legs were not measured. This pressure differential between the sides of the body is a medical fact that has been known for a long time, and was common for the medical practitioners, the home visits made by the physicians of the fore, to do so. It was also known that the method is not reliable - unless the pressure difference repeats itself for several times separated by several days, and the inventor does not know if the old-time physicians knew the reason for the method to be unreliable. The reason for the method to be unreliable is that the heart pumping strength, or pumping pressure, varies from heartBeat-to-heartBeat, which means that a later smaller blood pressure on the opposite side of the body could be due to atherosclerosis on that second side but it could be due to a weaker heart contraction while the measurement was taken as well! . . . This is why the method is unreliable, unless either 1) the pressure difference is LARGE, or 2) the pressure difference repeats itself over a period of time, over several visits by the physician. This known UNreliability of the method, which has been known, is one of the problems our amazing invention solves: with the interesting and unexpected changes disclosed in this patent application, the former unreliability will be gone, finito, no more, kaput—very good!

To complete our “prior art” (in attorneys old, stiff, funny words that makes no sense to the common person), or “existing devices” (in current language understandable by everyone), we describe how the blood pressure measurement with a sphygmomanometer works. For a medically trained person this part is already old stuff, so it at least can, and most likely should be skipped. This old-stuff part is the next three paragraphs. On the other hand, for a non-medically trained person, this part now is important to understand our invention. So, repeating, all medically trained persons already knows what follows in the next three paragraphs, which they could skip.

Blood pressure is a pair of numbers known as systolic and diastolic pressures, which is generally written as two numbers separated by a slash, as 122/78, for example, where the first number (122) is the systolic blood pressure (high), and the second, smaller number (78) is the diastolic blood pressure (low). The systolic blood pressure is the highest blood pressure applied by the heart on its pumping cycle, while the diastolic blood pressure is the lowest blood pressure applied by the heart on its pumping cycle, during the heart's resting phase. Most often the blood pressure is measured at the arm, just above the elbow, typically 1-2 cm up up from the elbow toward the shoulder, so, let us describe the process there, at the arm; the same method can be used at other locations, just changing the location of the cuff. Modern methods, e.g., the photoplethysmography devices, work differently and are capable of measuring the blood pressure at most places on the body, including at places not possible to measure with the sphygmomanometer. Our invention works with all the means to measure the blood pressure, as described. Finally, we are going to use the traditional blood pressure measurement with an inflatable cuff just above the elbow because it relates to what is today the most used method of measuring the blood pressure, even if for our invention the photoplethysmograph is preferable, for reasons that will become apparent later into this text.

The traditional way the blood pressure is measured is to put a cuff surrounding the location on which the blood pressure is to be measured, which, for us now will be the arm, just above the elbow, then to increase the pressure on the cuff, traditionally with a small hand pump, while looking at a pressure measuring device that measures the pressure at the cuff and at a device capable of detecting sounds originating from the blood flow through the vessels. The pressurized cuff squeezes everything inside it, including the arteries, where it is applied. If the cuff pressure is larger than the maximum heart pressure, then the artery is pinched, and the blood flow to the arm beyond the cuff location stops.

The medical person has the reading disc of a stethoscope under the cuff, and put the ear pieces on his ears. The stethoscope captures, and brings to its earpiece, the “puffing” sound of the artery suddenly opening to flow—not the flow, not the heartBeat, but the sudden opening of the artery that was pinched. This detail needs to be reiterated: the “puffing” is not the heart beat, as most people would imagine, but rather the “puffing” is the sudden opening of the artery, with the sudden blood flow when there was no blood flow before. This sudden “puffing” sound is technically called Korotkoff sound. There is no “puff” if (1) there is no artery pinching, because then the blood flow is continuous, and also there is no “puff” if (2) the heart is incapable of pushing the blood forward at that point because the artery is being pinched harder than what the heart can win—this is how the blood pressure is measured with the sphygmomanometer. The medical person who is measuring the blood pressure keeps pumping the cuff until the pressure measuring device indicates a value larger than the expected systolic pressure (high) for the patient, say, for a healthy patient, the medical person may pump up to 150 or 160, because this is larger than the systolic pressure for a healthy person. As soon as the air pressure created at the cuff is higher than the systolic (high) pressure from the heart, then the blood flow into the extremity of the arm stops, because the force caused by the heart squeezing is smaller than the force caused by the cuff squeezing the artery, and the stethoscope no longer detects any sound because there is no blood flow at all. From this high cuff pressure, the medical person starts slooooooowly decreasing the pressure at the cuff, until he finally hears one “puff”—the first sound of blood flowing; this marks the systolic (high) pressure, which is the reading at the pressure indicator, or pressure gauge PG at that moment. It is the pressure at which the heart can, finally, push blood through the arm at the cuff's location—the medical person will remember this number. As the medical person keeps releasing air from the cuff, therefore decreasing the pressure applied by the cuff on the arm, he will continue hearing the “puff” created by the blood flow breaching through the artery at each new heart contraction, because, the cuff being at a pressure higher than the heart minimum applied pressure throughout its pumping cycle, higher than the diastolic pressure, the flow does stop after the first “puff”, as the heart pumping pressure subsides. Then, as long as the cuff is at a pressure that is higher than the diastolic pressure (low pressure of the heart), the flow will stop when the heart pumping pressure reaches the continuously lowering cuff pressure. So the medical person keeps decreasing the cuff pressure and listening for the sound, until he no longer hears anything—no more “puff”! Some cuff pressure just below that last “puff” is the diastolic (low) pressure. If the medical person has been decreasing veeeeery slow, then the last “puff” may be actually the diastolic pressure, or else the diastolic pressure may be a little higher than the last “puff”. So, the medical person takes note of the cuff pressure when he hears the first “puff” on the stethoscope, which is when the higher pressure created by the heart contraction overcomes the opposing pressure created by the cuff, then again takes note of the last cuff pressure that causes the last “puff”, of the last sudden blood flow, which is the diastolic (low) blood pressure. Below the low pressure (diastolic) the cuff never stops the flow, which is then a continuous flow, no further “puffs”, because then the heart is applying a pressure that is never less than the cuff pressure. The main embodiment of our invention uses this sequence of events. To be precise here, the first “puff” is not really the systolic blood pressure, which could have been 1 or 2 units higher than the assigned number, depending on the speed at which the cuff's pressure is being lowered, because the assumed systolic is when there is the first opening, which could have occurred at a higher cuff pressure when the heart was in-between contractions, an event which was not probed. To be a correct measurement, the cuff's pressure should be lowered in such a way that the pressure at the cuff decreases by only one unit for every heart beat, this for both the high and the low values, for the systolic and the diastolic pressures.

Other methods of measuring the blood pressure may be applying the pressure at the cuff with an electric driven pump and/or using some type of non-human to detect the beginning of the opening of the artery at the systolic point, and detecting the end, when the artery is continuously open, at the diastolic point. The Japanese company Omron may have been the introducer of this method of measuring blood pressure without human intervention, or it may be only the manufacturer that is most known and used at this time, though many other brands are appearing now. This Omron device is capable of measuring the blood pressure of an animal, usually a person, then displaying the systolic and diastolic blood pressures, and also, usually, the number of heart beats per minute.

There are many models of automatic blood pressure measurements devices described in detail on the web, but not their technical internal details, and consequently we are not analyzing them in detail. The inventor does not know how Omron's device works. Omrom keeps it “seekret”, trade secret, as the attorneys say it!

Recapitulating, there are several methods to measure the blood pressure, as the traditional method with a sphygmomanometer and a stethoscope to hear the characteristic first and last “puff” (Korotkoff sound), while some artery is being under a decreasing pressure applied by a cuff, and more. Or the blood pressure can be measured with a photoplethysmography, that is, with measurements of light, perhaps of more than one wavelengths, depending on the model and method used. These methods are indirect; they usually do not measure the blood pressure, but other characteristics of the blood flow, usually the blood volume just under the skin, but may be other characteristics, or a combination of several characteristics, from which the blood pressure can be calculated using some algorithm.

### Objects and Advantages

Accordingly, it is an object of the present invention to detect internal obstructions on the arterial system, particularly on the subclavian arteries that supply blood to the arms, or on the internal iliac arteries, that supply blood to the pelvis and lower extremities, and on the external iliac arteries, that supply blood to the legs, on the subclavian artery, on the brachial artery, on the radial artery, and most other arteries carrying blood to the body of the animal, after the flow is already split to one side of the body or the other.

It is also an object of the present invention to pay off the mortgage of the inventors.

If one or more of the cited objectives is not achieved in a particular case, any one of the remaining objectives should be considered enough for the patent disclosure to stand, as these objectives are independent of each other, particularly the last objective.

### SUMMARY OF THE INVENTION*

Our brilliant invention discloses a method and a means to detect obstructions in the inner walls of pipes and other conduits that carry fluids from an origin point to a destination point. For our main embodiment we will use the blood as an example of a fluid, and the arteries as an example of the pipes/conduits, but the invention is intended to be applicable to any other fluid and any other pipe/conduits.

As described above, our device is designed to measure the blood pressure at two—or more, remember that it can measure the blood pressure at more than two locations—symmetric locations on the body of the subject, usually a human, which pressures are supposed to be the same if the subject is symmetric, which is the approximate case for most humans and even most other animals. In this case of symmetric build, at birth the arteries that go to the right and to the left are of the same internal diameter, the same wall thickness, the same stiffness, and so on. There are small variations at birth on each animal, small deviations from perfect symmetry, but, for most animals, including humans, the deviations are small and produce only small deviations of the measured blood pressure at two symmetric locations. If the dear reader wonder how much deviations from symmetry occur, he needs only to think about the deviations of symmetry on the external features of other people that he knows, as differences between the left and right eye, between the left and right ear, and so on; the differences are minimal to the point that, for most cases, unless one uses a machine to make measurements these differences would not be detected. On the other hand, if during the life of the animal some blood clots develop inside some artery, then the symmetry is no longer, it would be “kaput”, and the blood pressure may be different if measured on the left and right sides at the same location, say, just above the elbow on the left and the right arms.

This method of measuring the blood pressure at two symmetric locations on a human is known by the medical community, but it is seldom used, because, as stated above, they are masked by the error introduced by taking sequential blood pressure measurements, which then become vitiated by the variation of the heart strength of contractions, which varies from heartbeat-to-heartbeat. We need to stress this point here, because, though all physicians will state that yes, this is true, it is also true that most physicians do not have this clear in front of them before confronted with the statement: each heart beating is different in strength, or power, or compression, or pressure. In case that the reader doubts this last statement, just think of a boxer punching a bag: each punch is a little different in strength . . . Consequently, if a difference of the numerical value of the blood pressure is detected from two sequential measurements, the medical person is left with the problem of interpreting the difference as being a consequence of an arterial partial block or simple a consequence of the heart beating strength differences from the first to the second measurements. Usually the two usual locations to measure the blood pressure, are the bottom of the brachium, also said as the bottom of the upper arm, but any other pair of symmetric locations is possible and good.

After presenting the problem the way we did, the dear reader is likely to be already thinking:
“well, if the source of problem is the sequentiality of blood pressure measurements, why don't they simply make the blood pressure measurements at the same time, at the same cuff pressure?” If the dear reader thought it this way, then you anticipated the invention! It is a method that allows to keep the blood pressure exactly the same on two cuffs on two locations, and looking for the Korotkoff sounds on each location, as the common pressure is decreased together—see, e.g., FIG. 1, or FIG. 2, or a few others here.

Yes, our invention is a device that connects two symmetric locations with a tube, or any similar device, which forces that the pressure at both cuffs is always the same, for the same reason that the air pressure at one side of the room is the same as the air pressure at the other side of the room. Then, when the now same common pressure is decreased, it is decreased together at both sites, and from then on it all works the same as before! Very simple, very clever, very interesting and even fascinating. As a warning, we bring up front that with this method, if the blood pressure is measuring using the sphygmomanometer, then there is a need to have two medically trained data takers, each one with his own stethoscope, listening the sound at each one of the two cuffs, the left cuff and the right cuff. Alternatively, the two values of blood pressure can be detected automatically, using any of the available methods and means in use now, by Omron or by devices made by other companies.

The alert reader will have noticed that once the two cuffs are interconnected to force that the applied pressure is the same on both sides, then the “puff” (Korotkoff sound) should propagate from one side to the other, from one cuff to the other—with the potential of confusing the medical person listening for the Korotkoff sound! Indeed, given that the two cuffs are interconnected by a tubing, which function is to keep the same common pressure at both sides, it follows that the tubing does transmit sound as well, which then is a source of problems for the two separate medical persons (or any equivalent machine device) that is attempting to detect the “puffs” (Korotkoff sound). Good reader! :) The inventors also saw this, and disclose a sound dumper that is inserted along the interconnecting tubing, which sound dumper is capable of decreasing to an imperceptible level the “puff” (Korotkoff sound) propagating from one side to the other side, while allowing the two sides to be interconnected to force the same cuff pressure on both sides. An example of a sound dumper is the gun silencer, which is capable of substantially decreasing the sound of the firing, yet allowing the pressure to keep accelerating the bullet to its regrettable objective and function. But the inventor wants to highlight that the soundDamper is not necessary for the invention, but only an improvement on the invention. In fact, this being such a simple device, the inventor did purchase two sphygmomanometers, disconnected the hand pump from one of them, connect the now pumpless sphygmomanometer onto the other sphygmomanometer tubings, asked the help of a nurse (remember that two persons are required for the invention), and measured the result. The result was that yes, the inventor could hear the Korotkoff sound from the other side, but it was, as expected, much softer, easy to separate from the nearby Korotkoff sound. So, repeating it, the soundDumper is just an improvement to the invention but is not part of the invention, because the invention can work without it, as per the inventor's experience.

This link, from the Mayo clinic, states the same, only that there is no explanation for the steps: https://www.mayoclinic.org/diseases-conditions/high-blood-pressure/multimedia/how-to-measure-blood-pressure/vid-20084748

### BRIEF DESCRIPTION OF THE DRAWINGS

FIG. 1. Two blood pressure cuffs interconnected by a Y connector.

FIG. 2. Multiple blood pressure measurements—just air pump, no reading, no data, also no pressure equalization tube.

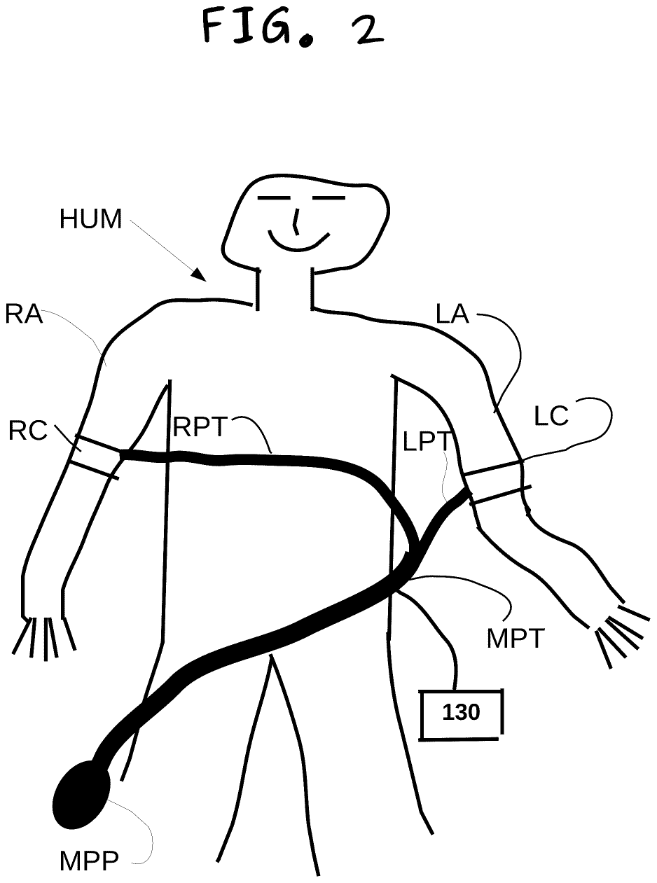

FIG. 3. Multiple blood pressure measurements, common manual pressure MPP and multiple human readers using the same common pressure gauge PG but SEPARATE stethoscopes St1R and St2L.

FIG. 4. Y-junction for pressure split.

FIG. 5. A T-connector to split the pressure created by the pump onto two branches. This is only one of the options for a spliter. See, e.g., the Yconnector.

FIG. 6. A Y-connector to split the pressure created by the pump onto two branches. This is only one of the options for a spliter. See, e.g., the Tconnector.

FIG. 7. Two cuffs connected by a tube located near each cuff.

FIG. 8. Definition of T_init for the main embodiment. Note that any other time along the heart contraction sequence can be used for T_init, this particular moment used for the main embodiment being not the only one for the invention to work.

FIG. 9. Right photoplethysmographer showing some of its innards.

FIG. 10. Left photoplethysmographer showing some of its innards.

FIG. 11. Left and right hands with photoplethysmography device clipped on the tip of the pointing finger.

FIG. 12. Conceptual representation of a Y-junction.

FIG. 13. Any pressure peaks spread out into HPD1, HPD2, HPD3, etc., with the result that the pressure at the exit hole, at the right side (gas flow out), contains less variations of the pressure peaks that may exist in the inlet pressure in, at the left hand side (gas flow in), which variations are the heart sounds. So, the pressure equalizer causes the pressure to be the same on both sides, while decreasing the heart sound crossings from one side to the other.

### DRAWINGS

### List of Reference Numerals

Ssuppr=Sound suppressor

ICU=intermediate controlling unit (ICU)

LC=left cuff. (cf. right cuff RC)

MP1=MPR=medical person 1, or right

MP2=MPL=medical person 2, or left

MPP=manual pressure pump, or main pressure pump.

PG=PI=Pressure gauge, also pressure indicator.

PPM=photoplethysmography devices.

RC=right cuff. (cf. Left cuff LC)

### DETAILED DESCRIPTION

### General Comments

Details of variation from main embodiment for arteries obstruction/atherosclerosis:

The physical structure of my wonderful invention is, in simple terms, more details starting next paragraph, two ordinary sphygmomanometer pressure measuring devices with the variations of (1) one tube or similar device to interconnect the two cuffs, keeping them at exactly and always at the same pressure, (2) one single pressure pump for two interconnected cuffs, and (3) one single pressure measuring device for the two interconnected cuffs. There is a need to detect two separate Korotkoff sounds, so, there is either two medically trained persons, capable of detecting the Korotkoff sounds from each separate cuff with a separate stethoscope each person, or else two automatic detecting devices of the type used, e.g., by the automatic machines manufactured and sold by Omrom or any of their equivalents.

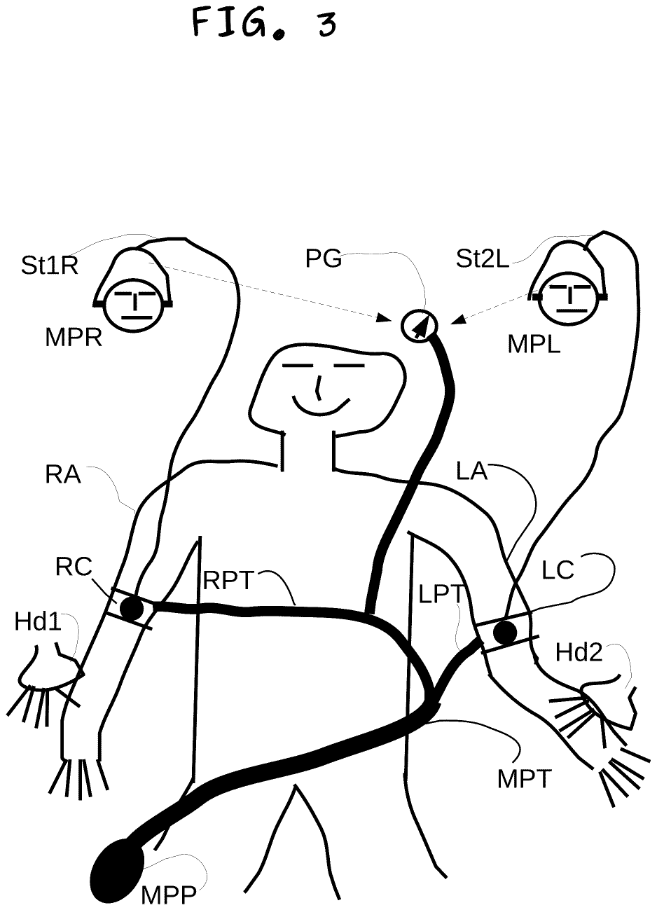

Figure FIG. 3 depicts a simplified view of our invention, but with the main parts visible and labeled. It shows two medically knowledgeable persons MPR and MPL (Medical Person Right and Medical Person Left), one at each side of the patient, who, for privacy, is smiling but not labeled in the figure. Each of the two medically knowledgeable persons MPR and MPL wears his own stethoscope that is inserted under the cuff at the side he is observing (right or left of the patient), labeled St1R and St2L, for Stethoscope 1 Right and Stethoscope 2 Left. There is a single pressure gauge reading device PG (for Pressure Gauge) or PI (for Pressure Indicator), which measures the common pressure at each cuff (right and left cuffs, RC and LC), which, being interconnected, are always at exactly the same pressure indicated by pressure gauge PG. Finally there is a main pressure pump MPP, which may be pumped by either MPR or MPL or the patient of another person (not shown) through the main pressure tube MPT that later bifurcates onto the right pressure tube RPT and left pressure tube LPT. The reader will notice that each of the two medically knowledgeable persons MPR and MPL looks at the same single pressure gauge PG, which displays the common pressure at both cuffs, RC and LC.

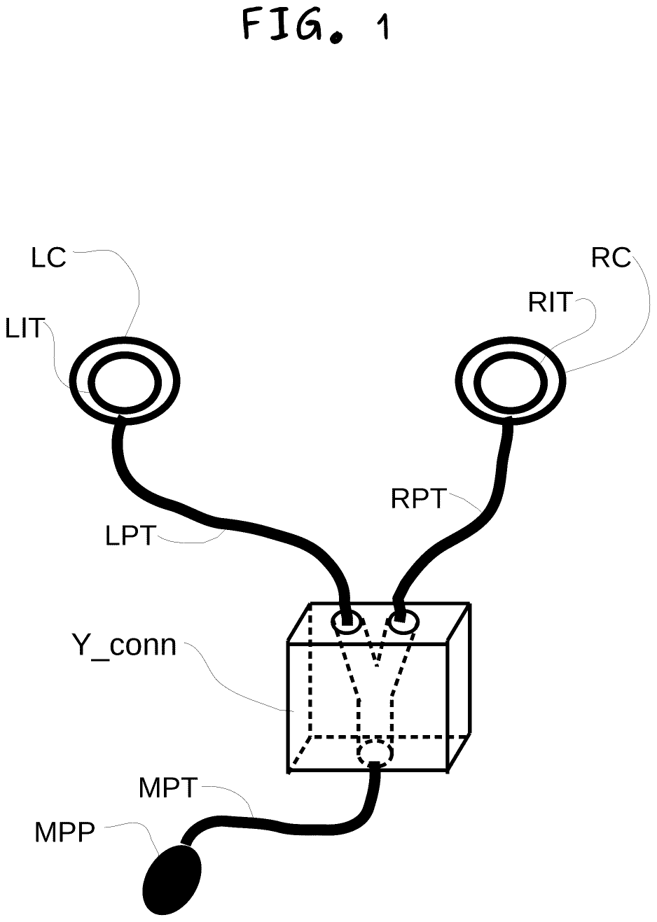

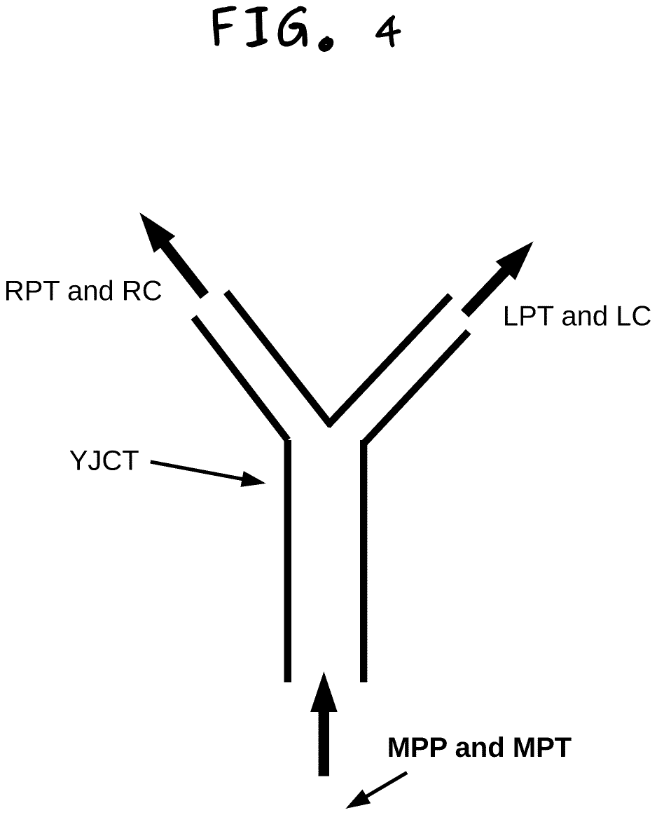

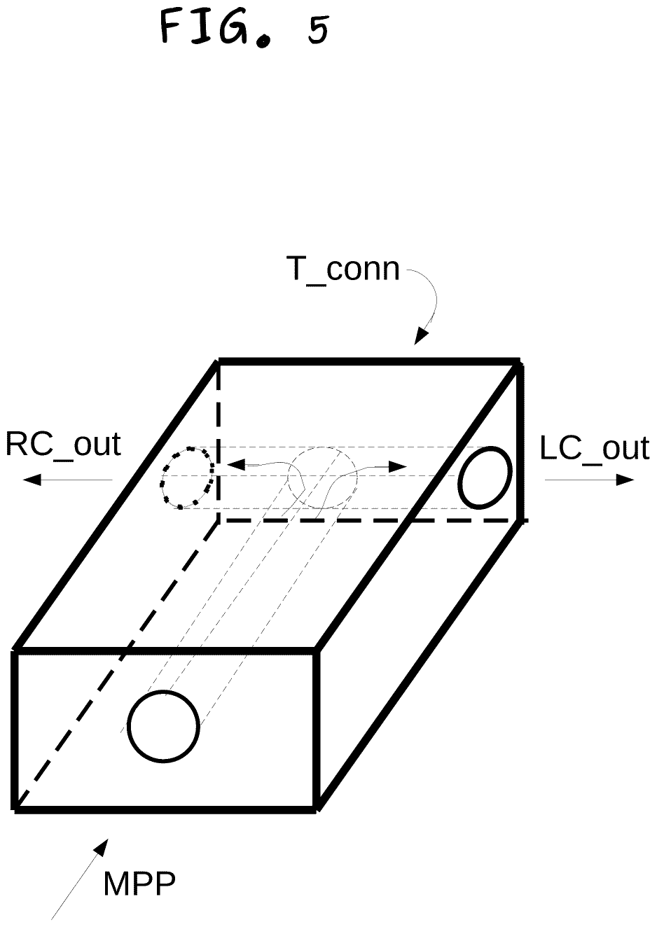

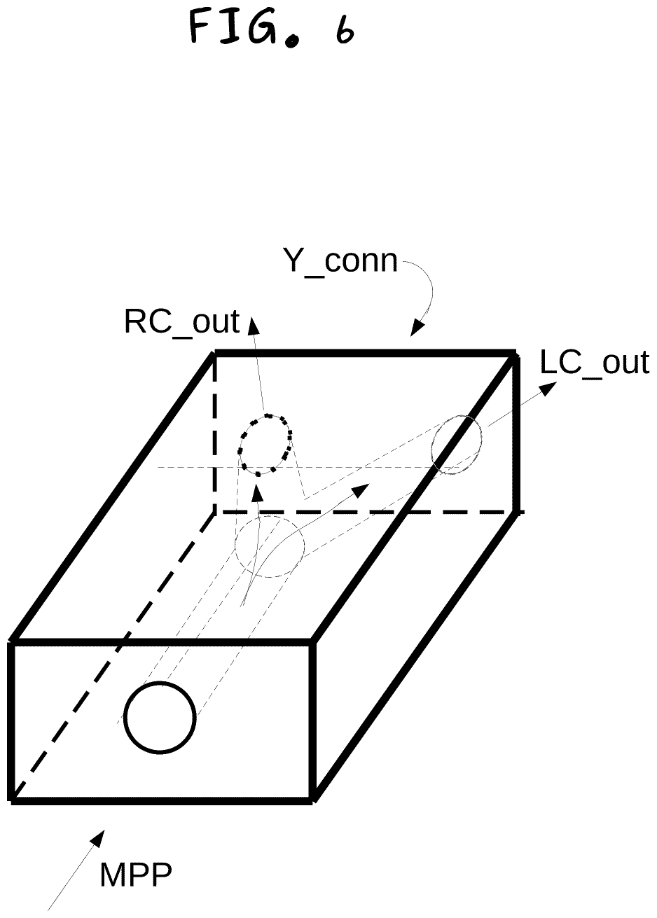

The main embodiment of our invention uses a tube splitting device similar to the ones shown at figures FIG. 1, FIG. 4, FIG. 5 and FIG. 6, but these are simply a few variations, out of zillions that are conceivable. Any elementary variation that any reasonable person, as a future member of a jury or a future judge, can see to be trivial, is to be considered the same thing as my beautiful invention. What matters for my invention is that all the elements are interconnected (1) the left cuff LC, (2) the right cuff RC, (3) the main pressure pump (MPP) and the pressure gauge (PG), which forces that the pressure on all of them is the same.

There are a number of simple variations on the above description of the hardware of our invention. For example, instead of two medically knowledgeable persons MPR and MPL, there could be two automatic devices, for example, similar to the devices used by Omron, which is the perhaps most known automatic blood pressure measurement device in use these days. Omron does not disclose, to my knowledge, how the device works, but it does not matter for the practice of the invention, which only needs something that measures the high and low values of the blood pressure—by any means necessary. Or, it could be a medically knowledgeable person and one Omron-type device. Or it could be measurements on three places, or four places, all these being a simple variation and/or extension of my invention.

Also, obvious deviations from my drawings and/or descriptions are to be considered as the same thing as my drawings.

My invention also discloses an improvement, which is not necessary for its workings, not necessary, but a simple improvement, which is the sound suppressor that prevents the “puffs” (officially called Korotkoff sounds), to go from one side to the other. Indeed, once the two cuffs are interconnected by a tube, the sounds travel from one side to the other, and consequently each medically trained person MPR and MPL hears the Korotkoff sounds from his side and also the Korotkoff sounds from the other side. Consequently there is an advantage in suppressing sounds from going from one side to the other. But, as the inventor observed, using the device, the Korotkoff sounds from the “other” side are substantially less audible—as expected—, and consequently our invention works without this sound suppressing element. We want to be explicit here, that the invention works, according to the experience of the inventor, without the sound suppressing device, so the sound suppressing device is not part of the main embodiment and not part of the independent claim. Moreover, the detection of the Korotkoff sounds may occur with a stethoscope, as suggested on the main embodiment, but the detection may be done by any other device capable of detecting it, as pressure detectors, electromagnetic detectors, and others, because for the invention all that matters is that the Korotkoff sound is detected by any means necessary.

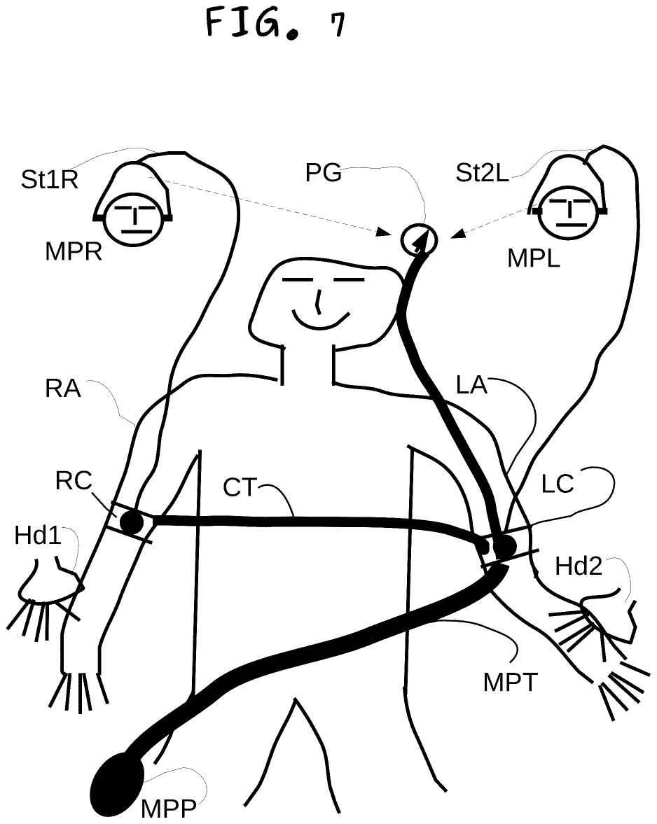

The inventor and writer here is a simple retired professor of physics, not conversant with the magic hand motions of the lawyers, so the inventor wants to describe the same thing again, with different words. Our invention is a device similar to, and potentially almost the same, as an ordinary, off-the-shelf sphygmomanometer, with a left cuff LC on the left arm, a main pump or main air pressure pump MPP, connected to the left cuff LC by a left pressure tube LPT, and (2) a second cuff, right cuff RC, which may be considered as another device similar to, and may be the same as a sphygmomanometer, but without the air pump and without the pressure measuring device, but only the right cuff RC part, on the right arm, so this is an extra and incomplete sphygmomanometer, and (3) a first pressure measuring device, or pressure indicator PI, and (4) a connecting tube CT, or pipe, or other similar conduit, which is located, in our main embodiment, near the cuffs connecting the right cuff RC to the left cuff LC. These are shown at figure FIG. 7. We call the attention to the dear reader that this latter, the connecting tube CT, or any of its equivalents, is the crucial part of our invention, the other, which is optional, optional, optional, not necessary, is the sound suppressor that prevents the “puffs” (officially called Korotkoff sounds), to go from one side to the other. The function of the connecting tube CT is to force that the two cuffs, RC and LC, are at the same pressure all the time, which is crucial to the working of our invention, as will be disclosed further down. Once the right cuff RC is connected by a tube to the left cuff LC, the two cuffs RC and LC are always applying the same pressure, for the same reason that the air pressure at a corner of the room where the reader is now is necessarily at the same air pressure of another corner opposite to it: if one place is at a higher pressure then the air would flow to the lower pressure location until the pressures were equal.

Recapitulating, this simple incarnation of the main embodiment uses two cuffs, a right cuff RC and a left cuff LC (FIG. 3) one on each arm of a patient who the medical person is screening for atherosclerosis. In one of the incarnations of the invention there are two cuffs, one each at each arm of the patient, perhaps a human patient, at the ordinary position that the cuffs are placed for the ordinary blood pressure measurements, at the upper arm, just above the elbow.

The two cuffs RC and LC are interconnected by a tube, preferably, but not necessarily, connected near each cuff, as shown in figure FIG. 3, but it is to be understood that the position of the interconnecting tube or tubes, with respect to each other and with respect to the whole thing are immaterial, it only being necessary that all the cuffs are connected by a tubing designed to make the pressure equal all over the whole damn thing.

### Examples of Intended Use

One example of intended use of our invention is to be implemented as a population-wide screening for arterial partial blockage. Such a population-wide screening cannot be done with the existing method of MRI, which is only available for the rich, but can be done with the device of our invention.

Another example of intended use of our invention is for individual preventive care medicine, similar to blood tests to detect the appearance of diabetes at an early stage, or to colonoscopy to detect a possible colon cancer at its early stages, or to mammography to detect a possible breast cancer at its early stages, or even the measurement of the blood pressure at the arm, which is commonly done, with the objective of start some dietary change or exercise program to bring down the blood pressure at its early stages, or even to take medicine to bring down the blood pressure. In our case, our invention allow the detection of atherosclerosis in the blood vessels.

Another example of intended use of our invention is to improve the quality of data collection about the health of the population.

Another example of intended use of our invention is to increase the reach of information about the health status of all patients with a simple device that is similar to the existing standard of measuring the blood pressure adopted so generally.

### L. Detailed Description

### Operation of Invention

My invention operates on, and can be understood by, some basic principles of fluid flow. Our invention is intended for any fluid flow, e.g., blood flowing in arteries and veins of an animal, a river, an oil pipeline, a gas pipeline, a water mains and smaller pipes inside a residential building, etc. The basic principle of operation is that for a fluid to flow in any container, including an artery, a pipe, etc., with with friction at the walls of the container (the artery, the vein, the pipe, etc.) the pressure at any position of the flow is always smaller at any point forward the motion than at any point before, along the motion of the fluid. Very simple, no big mathematics required, or stating it differently, the pressure on a conduit with a moving fluid and frictional losses of energy, always decreases along the fluid motion; it is always smaller at any and all points ahead when compared with any single position along the pipe that is before. Associated with this pressure decrease along the motion, as the fluid moves along, there is a concomitant decrease in speed—the fluid speed, as the fluid moves forward, is always smaller ahead, than it was before. An independent point of view for this decrease in the fluid velocity as it moves forward is that the friction with the walls of the pipe causes an energy decrease, which then manifests itself in smaller speeds of propagation, smaller kinetic energy. This assertions excludes the unusual case of superfluid, as helium-4, and of superconductors, both of which cases are excluded from our patent application.

The pressure at any point of a fluid in motion is, by definition, the applied force per unit cross-sectional area. A subtle point here is that the pressure in a fluid is applied along all directions, forward, backward, sideways, etc. Note that this is counter-intuitive, because it is not the case of isolated particles moving in a medium, as a car traveling along the road, etc. We want to emphasize this subtle point, because, as per experience of one of the inventors, most people do not see this, that the pressure on a fluid is applied along all directions, including backwards. Keeping this in mind, assuming that there are friction losses, which is virtually all cases, there are frictional losses on the fluid motion for the case of the main embodiment of the invention, which is blood flowing through arteries and veins. It is worth to note that the discussion here applies as well for oil flowing along an oil pipeline, or natural gas along a gas pipeline, or water along the water mains, etc., and our invention applies to all these cases equally well, and some of them are most important for engineering.

This pressure decrease on any fluid moving against friction on some pipe is a consequence of the energy loss against friction. Besides frictional losses to the wall of the container through which the fluid is flowing, other energy losses may occur, one of which are obstacles along the path of motion, as any decrease in pipe diameter, or any local occlusion (blood clots), or any bending of the pipe, and others. The main embodiment of our invention is the motion of blood in the arteries of some animal, perhaps a human animal, which arteries are subjected to internal deposits of fatty tissues (atherosclerosis) and other types of tissues on the inner walls of the blood vessels, arteries and veins and arterioles and venioles and capilaries, which deposits disturbs the blood flow, causing that energy is dissipated at these points, and an extra local decrease of the fluid pressure, which pressure drop propagates forward, because the pressure always keeps decreasing forward. It turns out that deposits of fatty tissues (atherosclerosis) inside the arterial walls is generally a health risk, so it is considered to be advantageous to detect this deposition of fatty tissues as early as possible, and, as it is the case nowadays, to clean the conduit up. We skip the discussion of how these deposits of fatty tissues, or any other type of tissues, affect the health of the animal, often decreasing his life span, referring the reader to the literature on the subject, both from the point-of-view of physics/engineering and from the point of view of medicine.

Therefore, the medical people is interested in discovering the existence of arterial occlusions as early as possible, because these occlusions are dangerous for the patient, also particularly important because now, since from a few years only, procedures have been developed to take these occlusions out, leaving a clean artery again. So it was normal that the medical people would have worked backwards the relationship arterial occlusions to blood pressure drop. Indeed, the latter, the blood pressure, is easy to measure, while it is difficult, complicated and expensive to make an image of the arterial occlusion, it being unrealistic to conceive making MRI images of the whole population. The above facts have been worked backwards by the medical personnel, who is interested in discovering the existence of occlusions in the walls of the arteries of his patient, and an unexplained drop in blood pressure is expected to be associated with some arterial occlusion.

Repeating our previous explanations, now from a different point of view, many variations of the main embodiment related is to use the difference in the blood pressure values or to the absolute numerical value of the blood pressure, when measured at symmetric locations on the body of the animal, or when measured at singular locations, as, for example, the tips of the right and left pointing fingers or any other location, can be useful to gather information regarding arterial occlusions.

A healthy human is expected to have the same value of blood pressure on each symmetrical location on his body, or at most some small differences, while another person that has a partial blockage in his right subclavian artery, or any other artery leading to the right arm, will have a blood flow to his right arm that suffered a sudden pressure drop at the location of the partial blockage at the particular right artery, which pressure drop propagates down stream, causing a lower blood pressure reading at the chosen measurement location anywhere along the arm, either (1) where the arm joins the forearm, just above the elbow, where the blood pressure is measured by the traditional, old method of sphygmomanometer, or (2) the tip of the pointing finger, perhaps measured by a photoplethysmography device, which may be larger than the small differences that occur due to small asymmetries on the body and therefore cannot be explained by any other than a partial blockage somewhere upstream from the lower blood pressure measurement.

It could become part of regular medical examination to measure not the blood pressure at the arm, but rather to measure the blood pressure at two locations that are symmetric on the body, the results of which measurements would be part of population screening for blood circulation problems.

### Introduction to a Second Mode of Operation of Our Invention

The main embodiment of our wonderful invention works on one of two modes, it being also possible to work with both modes together. The first mode is the one described above, which with the difference in the blood pressure at two symmetric locations on the body of the animal, which usually is a human, and the second mode, which we proceed to explain now, is with the time of propagation of the pressure wave through the arteries and veins.

Rephrasing the above comments from another point of view, for the reader to understand well the device of our invention, with the first mode of operation, and the first means, or device, what is of interest is the blood pressure differences when there exists a partial arterial blockage on one of the sides of the blood circulation. For example, if there is a partial blockage on the right subclavian artery, which brings blood to the right arm, not to the left arm, then the blood pressure from the partial blockage location on, downstream, is smaller at any location than it is at a symmetric location, which is on the left side, on this case of a partial blockage on the right subclavian artery. This is old stuff, everybody knows it and it cannot be patented. Our invention adds to this old matter the improvement of making the measurements at the same time, because sequential measurements, as it was previously done, fails to distinguish between variations due to the normal variation of each heart beating, and variations due to partial arterial blockage. Indeed, if the blood pressure is taken at the same time, then the heart squeezing is the same on both sides, because it is the same heart squeezing, and any differences can only be due to a partial blockage of the artery.

Therefore the first mode of operation of our invention solves the problem of disentangling the difference in blood pressure due to the beat-to-beat differences between each heart beat, and the difference in blood pressure due to a partial heart blockage. It is indeed well known in the medical community that a lower blood pressure on the right arm than on the left arm, may be due to a partial blockage on the artery leading to the right arm, say, the right subclavian artery, but it is also well known that the blood pressure difference is not accurate, because it may be due to differences of the heart strength of contraction from one beat to the other, and the fact that the blood pressures were traditionally taken in sequence. Our invention solves this old problem by the simple method—and the associated physical difference in hardware—of making the measurements at the same time, therefore with the same heart beatings. A note of explanation here is that most physicians have been trained to repeat that the left-right pressure measurements is an unreliable method to detect blood vessel occlusions (atherosclerosis), but they generally were never told why and they generally have no idea why the method is unreliable.

The second mode of operation, is based on the time of propagation of the blood through the arteries and veins. It is more compatible with the electro-optical devices known as photoplethysmographs but can be done with bare hands as well, even if not as perfectly. A device similar to the photoplethysmographs became ubiquitous with COVID-19, devices used to measure the level of oxygenation of the blood, but a slightly modified hardware can be used to measure the blood pressure. The method to make this measurement is well known and we are not describing it here, but simply refer the reader to any Google search and articles reading. On one of its versions, this second mode of operation of our invention it works either (1) measuring the elapsed time between a heart contraction and the arrival of the pressure pulse created by said heart contraction at one or more locations of the body of an animal that is downstream, along the blood flow, which may be compared with some known value of elapsed time that is reasonable for the patient, or (2) it works detecting any time elapsed between the blood pulsations on two symmetric locations on the body of the animal, which should be zero, pulsations at symmetric locations being at the same time, or (3) it works detecting the time elapsed between the blood pulsations on any two locations along the same propagation path, which is then compared with a known time expected, as previously determined from measurements on healthy subjects. Notice that option (2) is similar, but not the same, as the first mode described above, because the first mode uses the blood pressure difference, while the option (2) uses the time difference between two events. Also note that option (3) is essentially the same as option (1), with the only difference that (1) uses the starting time at the heart, which (3) uses the starting time somewhere else along the blood flow. Moreover, any device capable of detecting the heart contraction, or any other phase of the heart cycle, is compatible with the invention, and also any device capable of detecting a pressure pulse downstream from the heart compression is compatible with the invention.

On the second mode of operation, the device described in this patent application is intended to measure either (1) the absolute time elapsed between a heart contraction and the arrival of the effect of said contraction at some mid point or distant point on the body, or (2) to measure the relative time-of-event at two mid-position or extremity position on the body of the animal, usually a human, after the blood circulation has split in the two body sides, or (3) the relative time at events measured on two symmetric positions on the animal. The first case uses the measurement of the absolute time delay between a heart contraction and the arrival of the pulsation created by said heart contraction at, say, the arm, between the fore arm and the upper arm, where the blood pressure is usually measured, while the second case uses the time elapsed between the pulsation of the blood flow between two locations along a single pathway, and the third case uses time differences between two symmetric locations, say, at the right and left wrists. All are possible to use, the first can detect occlusions anywhere before the measuring point, including the initial part of the blood flow, with one single path, as the aorta, while the second can detect only occlusions located after the right-left blood circulation split.

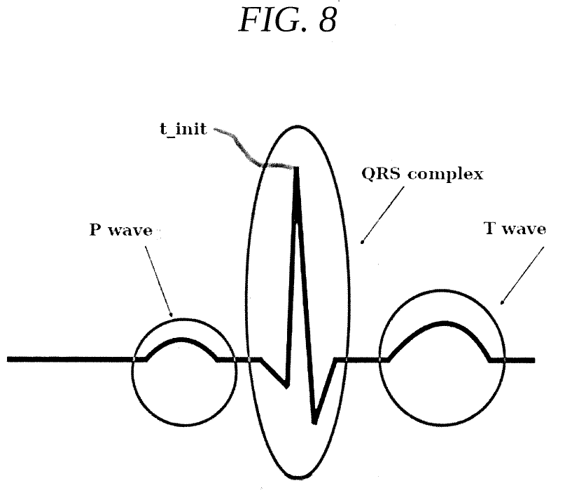

The delay time between said heart contraction and the arrival of the pulse pressure created by said heart contraction at distant locations on the body of an animal is a possible indicator of arterial obstructions, longer times being associated with internal arterial obstructions because the blood speed decreases at the obstruction location, the longer the time delay, the thicker should be the obstruction, or the longer, or the more repeatedly is(are) the obstruction(s), either of these increasing the time delay. The main embodiment discloses the use of electrical and/other method and means, e.g., sound and others, to detect the event of a contraction of said heart, and for the arrival of the associated pressure wave at distant locations on the body of the animal, it uses either the sound or the pressure or the electrical activity or any other means associated with the arrival of said pressure pulse at a distant artery, away from the heart. We will describe the main embodiment of our invention with an electrode similar to the electrodes used for, or similar to the EKG system, to detect the initial measurement time of a heart contraction sequence, with respect to which to measure the absolute delay from the heart contraction to the pulsation arrival at some distant location on the body of the animal, which usually is a human. An example of the initial time is shown at figure FIG. 8, where the initial time is the top R of the QRS complex, marked there as T_init, referred to a complete heart pumping cycle as measured by an ordinary, common EKG device.

Starting from the heart side, for a variation of the main embodiment of our invention we disclose the attachment of EKG-type electrodes to the skin of an animal, which electrodes are capable of making a necessary electrical contact with the skin such that an electrical potential originating from the regular contractions and relaxation of the heart of an animal can be transmitted from the skin of the animal to the electrode, where said electrical potentials are transmitted to wires. This may require the use of some conductive paste or conductive liquid, or the like, at the surface of said skin of said animal, and this and similar known variations in use for EKGs are included in our invention, yet the existing technology is not part of our invention, it being established knowledge, or “old art” as the patent attorneys call it with their pre-Shakespearean language. The electrodes are in turn connected to either electrically conductive wires or else connected to some at-a-distance communication, to some two-way wi-fi communication device, which is capable of using one of the several IEEE 802.11 standards, or a bluetooth standard, or any similar communication standard, any one is fine, which is capable of transmitting and receiving data from said sensor and transmit said data to said intermediate controlling unit or to some external equipment, etc. that are capable of transferring the electric potential from the electrode to some device capable of receiving said electric potential and measuring said electric potential that was captured by said electrode(s) at the skin of said animal, perhaps including the time variation of said electric potentials that originated at the heart. Our invention is not dependent on recording, or measuring, any time variation of said electric potential originating from the heart, but only needs to detect a time marker on said contraction, which time mark may be used as a reliable starting time for the time delay measurements, though a complete recording of the electrical signal is possible and may even be preferable. Said wires may be instead wi-fi connections (technically referred to as the IEEE 802.11) or any radio connection, or any other communication in use by electronic devices, without changing the nature of our invention, because our invention is not the communicating device per-se, but rather our invention is the clever use together of the information from the heart and the information from the pressure wave arriving at a distant location, and the method and means of acquiring said information and of using said information.

Continuing from the electrical conducting wires, as used for the main embodiment (or any other means of information transfer known and in use for electronics communication), the information of some time, any time, but a known and repeated time within the heart cycle P-QRS-T-P-QRS-T etc, on the heart contraction cycle, is transmitted to said intermediate controlling unit (ICU) or to said third communication device. For the main embodiment the Intermediate Controlling Unit (ICU) is a microcontroller, of the type of a Arduino, perhaps an Arduino-Duemile, but any other microcontroller would be fine, or even a tablet device, or a laptop device, or even a stupidfying-phone, wrongly referred to by the marketeers as a smartphone. The main embodiment of our invention transmits only and simply a particular time T_init within the heart cycle P-QRS-T, but it is equally possible to transmit the full wave form of the P-QRS-T cycle, leaving it for the Intermediate Controlling Unit ICU to decide what to use. This Intermediate Controlling Unit ICU receives electronic signals from the heart, and also receives electronic signals from the pulsating blood flow at one or more places on the body of said animal, preferably, but not necessarily, at some extremity of one or more of the limbs of said animal, e.g., one or more or the arms of said animal, or one or more of the legs of said animal, or the head of said animal, etc.

The Intermediate Control Unit ICU may be fitted with a time measurement device TMD, which may be a mechanical or electronic or any other type of time measurement device TMD, including an external time measurement device, for example a satellite standard clock, as used my many stupidfying Phone and many computers, etc. The main embodiment of the invention make use of an electrical/electronic time measuring device TMD. An example of an electronic time measurement device TMD is the famous 555 timer, a very old guy that all electronics enthusiasts know, but any other timer or any other oscillating circuit would be suitable for our invention as well, the 555 being not the only option possible. The main embodiment of our invention uses a 555 chip as the Time Measurement Device TMD. The Time Measurement Device TMD is used to measure the time elapsed between the arbitrary heart contraction time HCT, which is the initialTime T_init in FIG. 8, and any of the desired pressure pulse arrival time at any of the chosen extremity(ies). The initialTime T_init, which is a particular time within any heart contraction cycle is arbitrary, the main embodiment uses the maximum at the QRS complex, but may be any other instant of time within the heart contraction cycle without changing the invention.

Continuing from the Intermediate Control Unit ICU, electrical conductive wires or some form of radio communication connect the Intermediate Control Unit ICU to each of the extremity pressure arrival time sensors, which may be cuff-type sphygmomanometer pressure detectors, or, for the current version, photoplethysmography type detectors, or sound detectors, or any other sensor capable of detecting the arrival of the blood pressure wave at the location. Once this blood pressure wave is then detected by the extremity sensor, the time of the occurrence, which we call t_press (from time associated with a pressure pulse arrival) is used by the Intermediate Control Unit ICU, which allows the Intermediate control Unit, using the Time Measurement Device TMD and the InitialTime T_init to calculate the time elapsed between the heart contraction cycle arbitrary marker T_init and the blood pressure wave occurrence time t_press as the difference between them:

T_delay=T_press−T_init.

Therefore the delay time T_delay is the propagation time of the pressure wave, from the arbitrary initial time T_init to the arrival at any desired extremity time t_press, as a leg, or an arm, of the neck, etc. This delay time should be previously calibrated with a variety of patients, as the standard blood pressure is calibrated and a table of accepted healthy ranges and unhealthy ranges should be developed, as much as for it is for the blood pressure values.

At this point the reader can see it all . . . the time elapsed between the arbitrary time marker T_init and the blood pulsating time t_press at some distant point, is characteristic of each animal (a person, for example), as a consequence of his internal arterial diameters, his size (length of the arteries), and other characteristics that are particular of the animal (say, a person), but also changes with malfunctions of the blood circulation system, as atherosclerosis, etc, which is the goal of this patent application: to detect arterial occlusions. Before using the invention on patients, the device needs to be calibrated from a range of patients of a variety of heights, weights and other characteristics, patients known to have clean arteries, devoid from atherosclerosis, and a table compiled with the time delays t_delay according to the patient's characteristics, which later, in use, is used as the standard values to compare with the calculated delay on each test performed afterward. It is also possible, and a good practice, to calibrate each individual person while he is still healthy, for later comparisons of the time T_delay when the person ages and develop atherosclerosis and T_delay increases. If the time delay t_delay is larger than the normal, as indicated by the calibration table, then the patient may consider further examinations, as an MRI or something similar. The reader can appreciate that the calibration time is no different than the calibration time for the sphygmomanometer, the golden value 120/80 being numbers that resulted from observation on healthy patients, not from mathematical calculations; same as the future values resulting from the calibration for our invention.

Another possibility is the lack of simultaneity between two pressure pulsations on symmetric locations on the body of the patient, as on the left and right wrist—but any other pair of symmetrical locations is part of the invention. If there is some arterial obstruction on the right side of the body, after the bifurcation of the artery to the right and left subclavian arteries, then there occurs a pressure drop on the right side, which does not occur on the left side, with the concomitant speed decrease of the blood flow on the right side of the body, causing that the pulsation detected on the right wrist occurs after the pulsation is detected on the left wrist. This can be detected simply by any person with one thumb pressing the right radial artery (right wrist) and the other thumb pressing the other, the left radial artery (left wrist), but the electronic measurement allows for the medical person to know the extra time as a number, which allows for some sort of guess of the medical condition, as the severity, etc. The time delay t_delay can be also detected by our invention, which can give more information than a person testing both radial arteries with both thumbs, because our invention can measure the time delay, from which value the severity of the blockage can be estimated.

Here, differences in time between the arrival of a pulsating event on the right wrist and on the left wrist, most likely indicate an occlusion on the side with a later pulsating event, due to a smaller blood speed of motion inside the artery, because of the decreased propagation speed at the later side. Even if the two pulsating events are simultaneous, an occlusion may exist before the arteries split right-left sides, which can be detected by comparison with previously acquired data on animals (persons) of the same size, weight, etc. A moment of thought will show the reader that an occlusion before the split right-left would delay both sides equally, but would delay, which could still be detected if compared with the expected elapsed time for a person of the size-weight of the one under examination.

The reader has noticed that this mode of operation guarantees that the heart beats measured at the two symmetric locations are necessarily associated with the same heart contraction, and consequently the old difficulty with sequentially measuring the blood pressure at the right and left arms is automatically obviated. The photoplethysmographer is particularly suited for this operation, and since it can be applied at the extremities of the legs, say, at the toes, it can give information regarding arterial obstructions at the abdomen, because these arteries continue onto the legs and toes, which are very important because they are both more common and more dangerous as well, information that is absent from measurements at the arm or at the fingers.

### DETAILED DESCRIPTION

### Description and Operation of Alternative Embodiments

### Variations

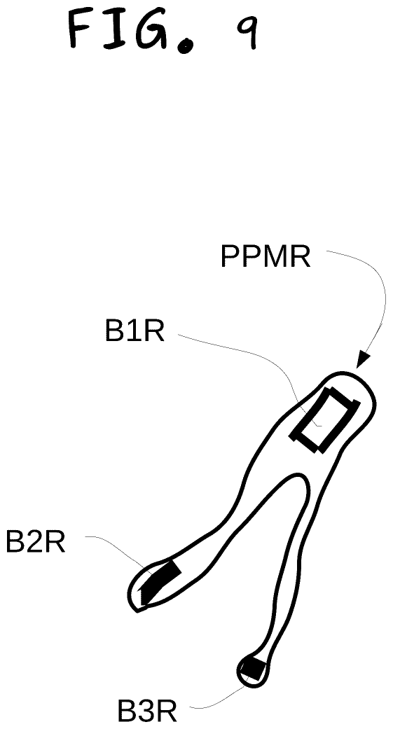

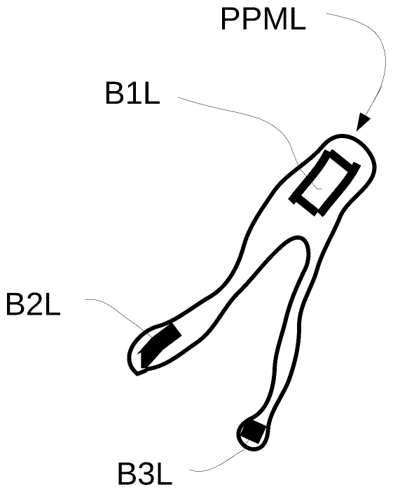

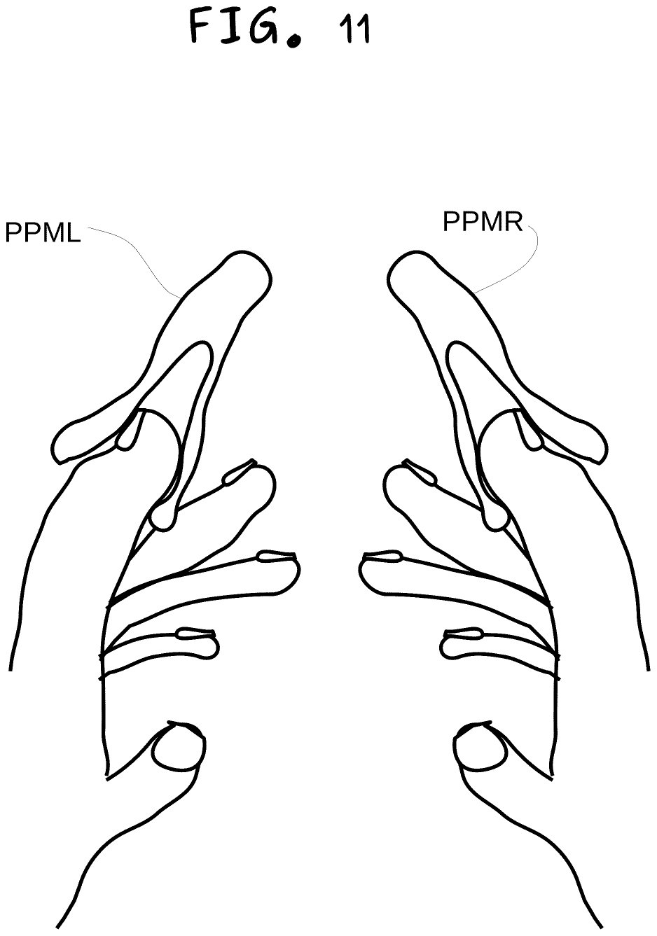

This is the detailed description for the photoplethysmography option of the invention. Figures FIG. 9 and FIG. 10 depicts an incarnation of the main embodiment of our wonderful invention, showing battery, electronics, light source(s) and light detector(s), which will be used for our discussion here, and figure FIG. 11 depicts a variation of it, this time including the hands of a person wearing the photoplethysmography device clipped on the tip of both the right and left pointing fingers. As shown at figure FIG. 11, the main embodiment of our invention, which is particularly geared for the detection of partial blockage of arterial blood, is composed of (1) a first photoplethysmography device PPMR attached at the fingertip of the pointing finger at the right hand of a person, which is adapted to measure at least the systolic and diastolic blood pressure values at the location where it is attached, (2) a second photoplethysmography device PPML attached at the fingertip of the pointing finger at the left hand of the same person, which is adapted to measure at least the systolic and diastolic blood pressure values at the location where it is attached, (3) a first two-way wi-fi communication device, which is part of B1R, the box that holds the battery and the electronics, connected to said first photoplethysmography device PPMR, which is capable of using one of the several IEEE 802.11 standards, or a bluetooth standard, or any similar communication standard, which is capable of transmitting and receiving data from a second two-way wi-fi communication device and/or a third two-way wi-fi communication device, which are both capable of using one of the several IEEE 802.11 standards, or a bluetooth standard, or any similar communication standard, (4) a second two-way wi-fi communication device, which is part of B1L connected to said second photoplethysmography device PPML, which is capable of using one of the several IEEE 802.11 standards, or a bluetooth standard, or any similar communication standard, which is capable of transmitting and receiving data from said first two-way wi-fi communication device, and said third two-way wi-fi communication device, (5) a third two-way wi-fi communication device (not shown) connected to said first and said second photoplethysmography devices, which is capable of using one of the several IEEE 802.11 standards, or a bluetooth standard, or any similar communication standard, this third two-way wi-fi communication device located at the location of said first photoplethysmography device PPMR, or at the location of said second photoplethysmography device PPML, or at another different location in proximity to the same person under examination. It will be obvious to persons familiar with the art of electronics that the number and locations of the communication devices, whether wi-fi or any other, can be different, and that they can be made in a single unit or on separate units, all technically possible variations being included in this description, this particularly composition above and other compositions below serving only as an example.

On the preferred embodiment the third two-way wi-fi communication device, using the communication standard, sends clock signals to said first and to said second two-way wi-fi communication devices, which clock signals said first and said second photoplethysmography devices use as the clock that keeps the local software running. Said first and said second photoplethysmography devices therefore receive the same clock signals from said third two-way wi-fi communication device, and consequently are synchronized to the same clock. Said first and second photoplethysmography devices, acting with the same clock signals received from said third two-way wi-fi communication device, have the capability of using existing technology to measure the blood pressure at their particular locations, which, in our preferred embodiment, is the tip of the pointing finger of the right and of the left hands of a human. The preferred embodiment of our invention uses any of the existing technologies to measure the blood pressure at the right and at the left finger tips of the pointing finger, with both photoplethysmography devices working together at the same clock signal provided by said third two-way wi-fi communication device, so that all the measurements taken by said first and said second photoplethysmography are taken together at exactly the same time. It is possible to skip the use of the common clock, allowing said first and said second photoplethysmography devices to work on different clocks.

On the preferred embodiment, the third two-way wi-fi communication device acts as a data collection center, and using the communication standard selected, receiving the data collected by said first and by said second photoplethysmography devices, which data said third two-way wi-fi communication device stores for later retrieval by some medical person or some of his authorized staff, for later analysis, or else for immediate analysis and perhaps repeat the data taking, or perhaps modifying the data request, another set of data and perhaps starting the process anew, as decided by the medical person or some other authorized staff. The third two-way communication device may incorporate some variation of microcontroller or some of its equivalents, and may act as an intermediate controlling unit (ICU) for the full device.

On the preferred embodiment, said first and said second photoplethysmography devices are capable of measuring not only the systolic and diastolic pressure of the patient on which they are attached, but also the full curve of the time variation of the values of the measured volume of blood and of the calculated blood pressures at their respective locations, and also to transmit said full curve of the time variation of the value of the blood pressures at their respective locations to said third photoplethysmography device. On the preferred embodiment said first and said second photoplethysmography devices also capture and send to said third communication device the full curve, as a function of time, of the amount of blood near the skin, as detected by the hardware of each. It is possible to implement the invention with less data as well, for example, it is possible to implement our invention with the collection of only said full curve of the volume of blood, as a function of time, of said amount of blood near the skin, as detected by the hardware or each the first and the second photoplethysmography devices.

On the preferred embodiment, said third photoplethysmography device is capable of transmitting all of the data it received from said first and said second photoplethysmography devices to a microcomputer, or to a tablet, or to a stupidfying phone (erroneously called smart phone by their manufacturers), or to a supercomputer, or to any other similar device. On the preferred embodiment, said third photoplethysmography device is also capable of carrying all the required mathematical calculations requested by the medical person, then display the results of said calculations in any one of a pre-selected number of formats.

On the preferred embodiment said first and said second photoplethysmography devices are off-the-shelf commercially available photoplethysmography devices, with an added also off-the-shelf electronics to be able to communicate using one of the several IEEE 802.11 standards, or a bluetooth standard, or any similar communication standard, or they may be specially designed photoplethysmography devices and/or specially designed communication devices.

On the preferred embodiment said third two-way wi-fi communication device is an off-the-shelf communication device, or is a specially designed communication device.

One possible variation from the main embodiment is attach said first and said second photoplethysmography devices at other locations not the tip of the pointing finger, as the tips of any other finger, or the wrist, or the arm, at the location traditionally used to measure blood pressure with a cuff, or at the ankle, or at the leg, or at any other location on the patient.

One possible variation from the main embodiment is to use a stupidifying phone in lieu of said third communication device (where the stupidifying phone is the accurate descriptor of any of the marketed devices referred to as smart phones, which they are not at all).

Another possible variation from the main embodiment is to use a microcomputer in lieu of said third communication device.

Another possible variation from the main embodiment is to use a specially designed microcontroller, as the 8085, or an Arduino, or any of its equivalents, in lieu of said third communication device. Or the third communication device may have several specific characteristics required by the device and also incorporate some type of microcontroller, as the 8085 or some of its equivalents. In some variations said third communication device may be called an intermediate controlling unit (ICU).

One possible variation is to use a stethoscope to capture the sound under or near the cuff, as it is usual for blood pressure measurements, with a microphone attached at the ear side of the stethoscope, to create the electrical signal for the intermediate control unit ICU. Such standard stethoscopes are located totally or partly under the cuff, or just near the cuff,

Another variation is a sound detector attached to one or both sides of the earpiece of the stethoscope.

Instead of a hand-pump device to pump air into the cuff, any type of automatic pump can be used, as electrical powered pumps, etc.

Another variation is to use a number of independent microphones attached to several locations on the skin of the animal, which microphones are capable of capturing the sound propagating at its location, then using said sound for analysis and discovery of location and size of obstructions inside the arteries and/or veins of said animal.

Another variation is to use a number of independent microphones attached to several locations on the skin of the animal, to detect abnormalities on the heart of said animal.

Another method for screening of atherosclerosis is the measurement of the blood pressure at two symmetric sides of the animal, e.g., measurements of the blood pressure at the right arm and the left arm. Indeed, a little thought would show that if there is some atherosclerosis on the right subclavian artery, the pressure drop at the position would propagate to lower blood pressures along the line, causing a lower blood pressure measured on the right arm when compared with the blood pressure measured on the left arm, both at the equivalent positions, usually just before the elbow - but any other pair of locations would be valid to screen for atherosclerosis before, or above, the particular point used. This method of looking for blood pressure at symmetric locations on the body of an animal has been used for a long time, more in the past than today, but still used today. There is a problem with using this method as it is usually done, which is that the blood pressure at the two symmetric locations are measured in sequence, one after the other, without considering that each heart contraction cycle is different, both the contraction strength, which causes the systolic blood pressure, and the later relaxing event, which causes the diastolic blood pressure. Consequently the pressure at which the blood starts to flow, having been squeezed by the cuff, depends on the particular heart contraction that occurs, which changes from one cycle to the next, so the sequential blood pressure measurements includes a natural variation. Our method changes the physical configuration of the pressure pump and of the cuff, causes that both cuffs are always at the same pressure, and decreases the common pressure together, which guarantees that each measurement of the blood pressure (say, right and left) is taken under the same physical environment. Forcing that the physical environment is the same on both sides at the same time, guarantees that the blood pressure measurement on each side is truly the correct value, and not a consequence of a stronger or weaker heart beating.

A different way to see, understand and construct a device that works and functions as our invention is a device similar to, and potentially the same as, an ordinary, off-the-shelf sphygmomanometer, with a left cuff LC on the left arm, a main pump or main air pressure pump MPP, connected to the left cuff LC by a left pressure tube LPT, and (2) a second cuff, right cuff RC, which may be considered as another device similar to, and may be the same as a sphygmomanometer, but without the air pump and without the pressure measuring device, but only the right cuff RC part, on the right arm, so this is an extra and incomplete sphygmomanometer, and (3) a first pressure measuring device, or pressure indicator PI, or pressure gauge PG, and (4) a connecting tube CT, or pipe, or other similar conduit, which is located, in our main embodiment, near the cuffs connecting the right cuff RC to the left cuff LC. These are shown at figure FIG. 7. We call the attention to the dear reader that this latter, the connecting tube CT is one of the main parts of our invention, the other part being the sound suppressor that prevents the “puffs” (officially called Korotkoff sounds), to go from one side to the other, but we stress that the sound suppressor is not necessary for our invention to operate and therefore it is not part of the invention but only an added extra feature. The function of the connecting tube CT is to force that the two cuffs, RC and LC, are at the same pressure all the time, which is crucial to the working of our invention, as will be disclosed further down. Once the right cuff RC is connected by a tube to the left cuff LC, the two cuffs RC and LC are always applying the same pressure, for the same reason that the air pressure at a corner of the room where the reader is now is necessarily at the same air pressure of another corner opposite to it: if one place is at a higher pressure then the air would flow to the lower pressure location until the pressures were equal.

Recapitulating, this simple incarnation of the main embodiment uses two cuffs, a right cuff RC and a left cuff LC (FIG. 7) one on each arm of a patient who the medical person is screening for atherosclerosis. In one of the incarnations of the invention there are two cuffs, one each at each arm of the patient, perhaps a human patient, at the ordinary position that the cuffs are placed for the ordinary blood pressure measurements, at the upper arm, just above the elbow.

The two cuffs RC and LC are interconnected by a tube, preferably, but not necessarily, connected near each cuff, as shown in figure FIG. 7.

The main embodiment of our invention receives information, or signals, from a detector at the middle of the right arm, above the elbow of said animal, at the position usually used to measure the blood pressure in humans. Said detector at the middle of the right arm of said animal is, for the main embodiment of our invention, a standard commercially available blood pressure measuring device, which is capable of detecting arterial pulsations at, or just above, the antecubital fossa, which is opposite to the elbow. Other variations and extensions of our invention uses detectors at both middle arms, and/or at one or both wrists, or some other position on the arm of said animal, or the arms and the legs, etc., all these variations being intended to be included in the invention, as they are obvious variations and extensions of the main embodiment with one single detector at the right middle arm of said animal.

### Conclusion, Ramifications, and Scope of Invention

The intermediate controlling unit ICU used in our invention may be an Arduino Duemile, but any other microcontroller-type device would serve equally well, for example, a Raspberry Pi, a BeagleBone, a Sharks Cove, a Minnowboard MAX, a Nanode, a Waspmote or an LittleBits, or any similar device, including the simple microprocessor or microcontroller, as an 8080, or an 8085, etc., or a tablet reader, or a notebook computer, or a desktop computer, or a supercomputer, and many of their equivalents. In particular, a stupidfyingPhone, wrongly called smart phone, would be most suitable, given its ubiquitousness.

We used the sphygmomanometer for the main embodiment of the invention, but there are many other devices that work to do the same. Any device that is capable of measuring blood pressure at the two selected locations is acceptable. For example, there are blood measuring devices that uses light, or other electromagnetic radiation, which are variations and extensions of the FitBit, or of the oximeter, and the like, which may also be employed to measure the blood pressure at two locations, with the objective of determining arterial partial blockages, with modifications to adapt to each method and each means and each device. Other possibility is to use the invasive blood pressure detectors that are often used during surgery, which the anesthesiologist uses to verify the state of the patient. It is possible to use two symmetrical detectors, connected to a central receiving data collection, perhaps an intermediate control unit, or any microcontroler or the like, which can compare the measurements (the numbers) from each detector then either display an interpreted result or else display all the accumulated data, perhaps, for example, two curves displayed on the same graph, or a spreadsheet or any similar table with the numbers collected at regular intervals, say, every millisecond, or every ten milliseconds=0.01 s, or every 100 milliseconds=0.1 s, etc. The type of blood pressure detector does not matter. Our method is usable with any blood pressure detector.

For the photoplethysmography devices it may suffice to capture the time variation of the blood supply just at and/or below the skin surface, then use some of its points, e.g. a point-of-inflection or any other, to determine a known time or pulse arrival, which can be compared with the equivalent one on the symmetric location, or with some of the initial time as chosen, e.g., T_init. This would avoid having to calculate the blood pressure value from the curve of blood supply vs. time, which is normally obtained by most photoplethysmography devices.

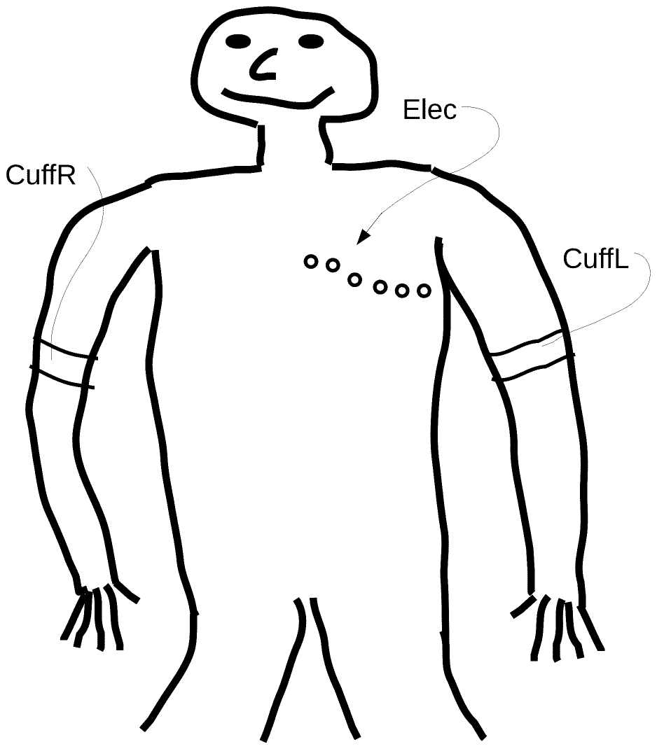

Some variations of the photoplethysmograph may use a full or a partial set of electrodes of the type used by EKG (ElektroKardioGram), as shown on FIG. 12. Variations of the electrodes may be used for our invention.

Said first and second photoplethysmography devices, which, on the main embodiment are attached to the fingertips of the pointing finger of the person under examination, may be located at other positions, e.g., the tip of some other finger, or the tip of any of the toes, or the wrist, or the middle of the forearm, or the upper end of the forearm, where it joins the arm, near the elbow, or at the lower part of the arm, near the elbow, or at the upper part of the arm, near the shoulder, etc. Any position on the body of the person under examination is possible to host the measurements by the first and second photoplethysmography devices.

The reader will understand that instead of using two photoplethysmography devices to measure the blood pressure at two or more locations, it is possible to use any other method, for example, to use one of the available oximeters, and/or one of the available FitBits, and/or any of the other devices that uses light, or infra red “light”, or two sphygmomanometers, or an invasive probe that may be inserted in the artery, usually by the anesthesiologist, during surgery, to continuously measure the blood pressure, or one of the many other possible methods to measure the blood pressure. The reader will also see that it is possible to use one of the commercially available automatic, self-running blood pressure measuring devices, many of which use the sound that propagate through the same tubing used to inflate the cuff, or any other method. The reader hopefully can see that the particular method to measure the blood pressure is not important, but that our invention detects the existence of partial arterial blockages from the difference in blood pressure, and that it does not matter how the blood pressure is measured, but only that the difference in blood pressure numerical values is detected. The dear reader will also understand that it is not necessary to actually measure the blood pressures, but simply the curve of the blood supply, as a function of time, which is generally the raw data obtained by most photoplethysmography devices.

For the main embodiment, said photoplethysmography devices are located at the tip of the pointing fingers, but a possible variation of the main embodiment may use the traditional devices used to measure blood pressure, called sphygmomanometers instead. On this variation from the main embodiment, the detectors are located preferentially, but not exclusively, at the middle of the right arm of said person, or patient, or said animal, and the sound detector may be either a stethoscope controlled by a medical person, or may be a sound detector, as a microphone, which may be either directly attached to the skin, in direct contact with the patient, or is coupled to an ordinary stethoscope. Said stethoscope is positioned, for the main embodiment, at the usual location where stethoscopes are positioned to measure blood pressure at the middle arm of a human: under a cuff that is connected to a pressure applying device, which is also capable of slowly, and under the control of a medical person, to decrease the pressure applied by the cuff. Said sound detector is capable of creating an electric signal proportional to the amplitude of the sound produced by the arrival of the pressure wave at the middle of the right arm. Said electric signal from said sound detector is transmitted to said intermediate controlling unit ICU, or else transmitted to to said third communication device, which records the time of the arrival of said electric signal from said sound detector, which we call t_2. Said intermediate controlling unit has electronic circuits that allows it to display this arrival time t_2. The display of time t_2 may be within the same display used for T_init, or may be a separate display, it only matters that both times are available for reading and later processing. If T_init is displayed on an ordinary computer monitor, then it is possible that the same said ordinary computer monitor displays the numerical value of t_2, while if both T_init and t_2 are displayed on a seven-segment display, then either number is displayed separately - both numbers, T_init and t_2 need to be available.

The difference between the numerical values T_init and t_2 is the time elapsed from a known event within the heart pulsating cycle and the arrival of the pressure pulse at the middle of the right arm of said animal, let us call this time difference propagation time t_propag:

t_propag=T_init−t_2

The propagation time t_propag is the time it takes for the effect of the heart pumping to appear at the middle of the right arm of said animal, which time can then be compared with a pre-determined normal time that the effect of the heart pumping to appear at the middle of the right arm of said animal. Said normal time is a range of values, perhaps depending on the size of the animal, perhaps on the length of the arm, and other factors that are not part of the invention, but are part of the calibration of the system to apply the system to different sizes of the animal, also to different body types, as weight, etc., none of which is part of our invention, which is only the method and the means. As long as the value of t_propag is within the normal values determined by calibration with healthy animals, it means no arterial obstructions, but if t_propag is larger than the normal values determined by calibration, than the poor animal has a problem! Our invention is the method and the means to detect, from the measured longer times t_propag, the existence of a possible obstruction inside the arteries, when compared with a normal time value obtained from calibration using a variety of animals that is capable of cover the necessary variations to compare with the measurement at an animal which is under examination. The animal is usually a human, but it can be any animal.

Another aspect of our invention is the electronics unit that is capable of implementing choices on the device, which we call the intermediate controlling unit ICU. This intermediate controlling unit ICU, also called third communication device may be an Arduino Duemile microcontroller, but any other device with equivalent capabilities is possible, the Arduino being used as an example only, and other possibilities are possible, for example, a tablet, a desktop microcomputer, a notebook computer, a similar microcontroller, as an 8085-based microcontroller, or a stupidfyingPhone, which is wrongly called by the name smartPhone, and many other similar devices.

The description at the specification that describes two cuffs at symmetric locations, e.g. left-and right-arms, above the elbow, which is the standard location to measure blood pressure, may be extended to three, four, or more cuffs at as many locations. Prior calibration would teach the medical professional how to compare the blood pressure at the leg, above the knee, and at the leg, above the anckle, and other locations, with the blood pressure at the arm, just above the elbow and among each of them.

Regarding to the blood pressure going to the head, say, the blood pressure measured at the neck, the cuff method and Korotkoff sound may be problematic, because even a short decrease in blood supply to the brain may cause the animal to faint, but the blood pressure at the neck can be “measured” by some modern methods that do not block the blood flow, as the photoplethysmographer, for example, or some other method.

The main embodiment for Photoplethysmographer style uses the Photoplethysmographer at the tip of the fingers of the patient. This would be good to detect atherosclerosis or any blood flow obstructions on the arteries leading to the hand, which includes the arteries at the abdomen, which are known as a big widowMaker when they burst. A similar result is obtained locating the Photoplethysmographers at the big toe, left and right, for example. In this alternative location the device of our invention would be detecting blood flow obstructions on the abdomen and below, which are actually more prevalent and more dangerous. In general, the Photoplethysmographer can be located at any pair of locations, or any single location, to measure a set of possible blood obstruction sites. As an example we also mention the use or our invention to measure blood flow obstructions just before and at the head of the patient, with detectors located just before the head (say, the neck) or at the head.

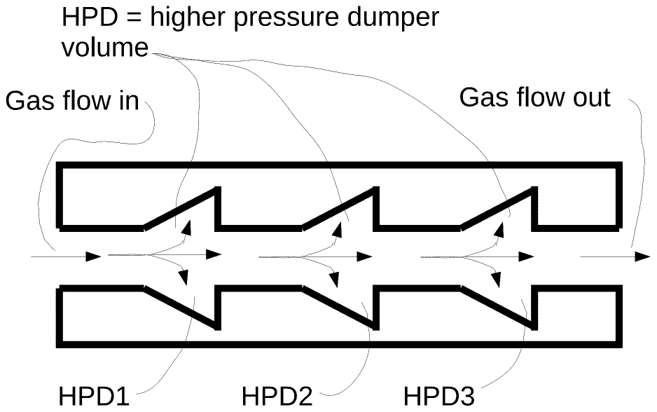

Finally, there is a possible improvement, which is not part of the main embodiment because our invention does work without it, the the sound suppressor, the equivalent to the gun silencer mentioned above and shown at FIG. 13. FIG. 13 shows a sound suppressor that is virtually identical to an ordinary gun silencer, but other variations are covered in our invention.

### SEQUENCE LISTING

N/A

What is claimed is:

1. An apparatus for detecting obstructions inside conduits inside which there occurs a flow of a fluid, said flow moving inside multiple paths of said conduits, inside which said conduits said fluid propagates under the influence of a first pressure pump located at a first location, said apparatus having:
a minimum of two cuffs called first cuff and second cuff,
a second pressure pump adapted at applying a pressure at said minimum of two cuffs, said second pressure pump being adapted at increasing the pressure on said minimum of two cuffs and also adapted at decreasing said pressure at a desired rate of decrease, causing a pressure change on time,
at least one tubing interconnecting said minimum of two cuffs,
said minimum of two cuffs adapted at causing obstructing pressure on said conduits on at least one point on at least one of said multiple paths of said conduits,
a gauge adapted at measuring and displaying the value of the pressure at said minimum of two cuffs as said pressure at said cuffs changes on time,
a minimum of one device adapted at detecting the first and the last Korotkoff signals while allowing a method to associate a first pressure reading to the first Korotkoff signal and to associate a second pressure reading to the last Korotkoff signals.

2. The apparatus of claim 1 where said device adapted at detecting the first and the last Korotkoff signals is a minimum of one stethoscope.

3. The apparatus of claim 1 where said fluid is blood and said conduit is either an artery or a vein.

4. The apparatus of claim 1 where said obstructions are atherosclerosis deposits inside said arteries.

5. The apparatus of claim 1 where said obstructions are blocked from direct observation to form visual images.

6. The apparatus of claim 1 where the pressure measurements are obtained with the aid of a photoplethysmography device.

7. The apparatus of claim 1 where said at least one tubing interconnecting said two cuffs is adapted at keeping the pressure at said minimum of two cuffs to be the same pressure.

8. An apparatus for detecting obstructions inside a conduit, inside which a fluid propagates under the influence of a pump located at a first location, said detecting occurring from the measurements of the time of propagation of a pressure wave, from said first location of said pump to a second location, such that said second location is downstream, away from said pump along the direction of the fluid flow.

9. The apparatus of claim 8 where said fluid is blood and said conduit is an artery or a vein.

10. The apparatus of claim 9 where said obstructions are atherosclerosis deposits inside said arteries.

11. The apparatus of claim 8 where said obstructions are blocked from direct observation to form visual images.

12. The apparatus of claim 8 where the pressure measurements are obtained with the aid of a photoplethysmography device.

13. A method for detecting obstructions inside a conduit, inside which a fluid propagates under the influence of a pump located at a first location, said detecting occurring from the measurements of the differences in fluid pressure values at two symmetrical locations along the propagation of said fluid when said conduit splits into two sides that are similar in characteristics.

14. The method of claim 13 where the fluid is blood and the conduits are arteries and the pump is a heart.

15. The method of claim 14 where the measurement of the value of the blood pressure is measured with a sphygmomanometer.

16. The method of claim 14 where the measurement of the value of the blood pressure is measured with an optical device.

17. The method of claim 14 where the measurement of the value of the blood pressure is measured with an photoplethysmography device, a fitbit device, or an oximeter device.

18. The method of claim 14 where the measurement of the value of the blood pressure is measured with a SmartWatch device.

19. The method of claim 14 where the measurement of the numerical value of the blood pressure is measured with an invasive pressure detector inserted in an artery.

20. The method of claim 14 where the measurement of the numerical value of the blood pressure is measured with an Omron type of blood pressure measuring device.
